(19) United States
(12) Patent Application Publication
(10) Pub. No.: US 2014/0296520 A1
(43) Pub. Date: Oct. 2, 2014

(54) PROCESS FOR THE PREPARATION OF VALACYCLOVIR HYDROCHLORIDE

(71) Applicant: PIRAMAL ENTERPRISES LIMITED, Mumbai (IN)

(72) Inventors: Mita Roy, Mumbai (IN); Ashutosh Jagtap, Mumbia (IN); Nitin Pawar, Mumbaia (IN)

(73) Assignee: PIRAMAL ENTERPRISES LIMITED, Mumbai (IN)

(21) Appl. No.: 14/358,179

(22) PCT Filed: Nov. 23, 2012
(86) PCT No.: PCT/IB2012/056649

§ 371 Date: May 14, 2014

(30) Foreign Application Priority Data
Nov. 25, 2011 (IN) 3323/MUM/2011

(51) Int. Cl. C07D 473/18

(52) U.S. Cl. CPC C07D 473/18
USPC 544/276

(57) ABSTRACT

The present invention provides a process for the preparation of 2-[(2-amino-1,6-dihydro-6-oxo-9H-yl)]ethyl L-valine ester hydrochloride (valacyclovir hydrochloride) of formula I comprising deprotection of N-[(benzyloxy)carbonyl]-L-valine-2-[(2-amino-1,6-dihydro-6-oxo-9H-purin-9-yl)methoxy]ethyl ester, of formula II using 5% palladium on carbon as a catalyst and mineral acid in the presence of water, avoiding use of organic solvents under hydrogen pressure to yield valacyclovir hydrochloride having yield of ≧90% and purity of ≧99.5%, pharmaceutically acceptable grade. The valacyclovir hydrochloride obtained using the process of the present invention is valacyclovir hydrochloride polymorphic Form I.

### FIELD OF THE INVENTION

The present invention relates to a process for the preparation of 2-[(2-amino-1,6-dihydro-6-oxo-9H-purin-9-yl)methoxy]ethyl L-valine ester hydrochloride, (valacyclovir hydrochloride), represented by formula I. The present invention further relates to an industrially applicable process for the preparation of valacyclovir hydrochloride.

### BACKGROUND OF THE INVENTION

Valacyclovir hydrochloride, 2-[(2-amino-1,6-dihydro-6-oxo-9H-purin-9-yl)methoxy]ethyl L-valine ester hydrochloride, structurally represented herein below by formula I, is an oral antiviral drug, which is used in the treatment of genital herpes and herpes zoster.

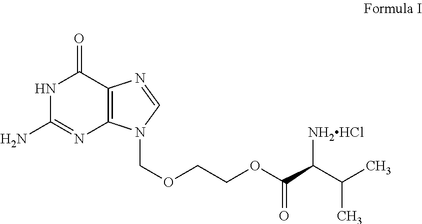

Valacyclovir is a prodrug that is derived from acyclovir by esterifying 3′-hydroxyl group of acyclovir with L-valine. Acyclovir is an antiviral nucleoside that possesses activity against human herpesviruses. Owing to its limited bioavailability, acyclovir has shown moderate antiviral efficacy after oral administration. Valacyclovir hydrochloride is not active itself, rather it gets converted in-vivo to acyclovir which is active against the herpesvirus.

The oral administration of valacyclovir hydrochloride is advantageous than that of acyclovir because acyclovir is poorly absorbed from the gastrointestinal tract. In contrast, valacyclovir hydrochloride is rapidly absorbed from the gastrointestinal tract after oral administration and is converted to acyclovir and L-valine. Valacyclovir has been reported to increase the oral bioavailability of acyclovir by 3- to 5-fold in humans (Antimicrob Agents Chemother.: 1995 December; 39(12):2759-64). Valacyclovir hydrochloride is available in the market under the trade names Valtrex® or Zelitrex®.

The process for the preparation of valacyclovir hydrochloride of formula I has been reported in several prior arts. Generally, the process involves deprotection of N-[(benzyloxy)carbonyl]-L-valine-2-[(2-amino-1,6-dihydro-6-oxo-9H-purin-9-yl) methoxy]ethyl ester (the compound of formula II) using a reducing agent such as, palladium on carbon, palladium on aluminum oxide and mineral acid in the presence of organic solvent or a mixture of organic solvents and water to obtain valacyclovir hydrochloride.

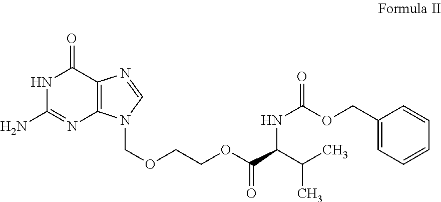 [SMILES: CC(C)[C@H](NC(=O)OCc1ccccc1)C(=O)OCCOCn1cnc2c(=O)[nH]c(N)nc21]

Valacyclovir and its pharmaceutically acceptable salts are disclosed in U.S. Pat. No. 4,957,924 (hereinafter referred to as US'924 Patent). The process for the preparation of valacyclovir hydrochloride described in US'924 Patent comprises deprotection of N-[(benzyloxy)carbonyl]-L-valine-2-[(2-amino-1,6-dihydro-6-oxo-9H-purin-9-yl) methoxy] ethyl ester (the compound of formula II) using 5% palladium on carbon, as a catalyst in the presence of 26.8 volumes of methanol, 26.8 volume of tetrahydrofuran (THF) and 4.8 volume of 0.5M hydrochloric acid solution at hydrogen pressure of 50 psi for one day. The reaction mixture is then filtered to obtain a white solid. The solid obtained is then recrystallized from water/ethanol to yield 60% valacyclovir hydrochloride. The process described in the U.S.'924 Patent requires use of a large volume of organic solvents. Also, the disposal of such large volume of organic solvents or mixture of the organic solvents as effluent in commercial manufacturing of the compound is cumbersome. Thus, the above discussed process for the preparation of valacyclovir is industrially not feasible.

U.S. Pat. No. 6,107,302 (hereinafter referred to as US'302 Patent) discloses a process for the deprotection of N-[(benzyloxy)carbonyl]-L-valine-2-[(2-amino-1,6-dihydro-6-oxo-9H-purin-9-yl)methoxy]ethyl ester, of formula II using 5% palladium on carbon in the presence of 4.5 volumes of denatured alcohol and formic acid to obtain valacyclovir. The resulting valacyclovir on further treatment with hydrochloric acid and subsequent purification using acetone yields 87.6% valacyclovir hydrochloride. The process described in US'302 Patent requires use of an organic solvent and a mineral acid for the deprotection of said compound of formula II to yield valacyclovir hydrochloride. Also the use of formic acid as a source of hydrogen generates impurity 2-[(2-amino-1,6-dihydro-6-oxo-9H-purin-9-yl)methoxy]methyl N-formyl L-valine ester (N-formyl impurity).

Also, the use of alcohol such as methanol or ethanol as the reaction solvent generates the impurities namely, 2-[(2-amino-1,6-dihydro-6-oxo-9H-purin-9-yl)methoxy]ethyl N-methyl-L-valine ester (N-methyl impurity) and 2-[(2-amino-1,6-dihydro-6-oxo-9H-purin-9-yl)methoxy]ethyl N-ethyl-L-valine ester (N-ethyl impurity), thereby rendering the process disadvantageous.

Indian Patent Application No. 2521/MUM/2007 describes a process for the preparation of valacyclovir hydrochloride comprising deprotection of N-[(benzyloxy)carbonyl]-L-valine-2-[(2-amino-1,6-dihydro-6-oxo-9H-purin-9-yl)methoxy]ethyl ester, of formula II using noble metal catalyst in the presence of mixture of methanol and hydrochloric acid as a solvent to obtain valacyclovir hydrochloride.

Indian Patent Application No. 465/MUM/2006 discloses a process for the preparation of valacyclovir hydrochloride involving deprotection of N-[(benzyloxy)carbonyl]-L-valine-2-[(2-amino-1,6-dihydro-6-oxo-9H-purin-9-yl)methoxy]ethyl ester, of formula II using palladium on carbon in the presence of methanol and hydrochloric acid to obtain valacyclovir hydrochloride.

The process for the preparation of valacyclovir hydrochloride, the compound of formula I can be improved particularly in terms of cost, by providing simple, efficient and industrially applicable process for the preparation of the compound that would result in good yield and purity of valacyclovir hydrochloride. The processes for the preparation of valacyclovir hydrochloride described in the cited prior art references mostly involve use of large volumes of organic solvents, which involves the issue of disposal of the waste effluent that may be generated during commercial manufacturing. Also, the purification of valacyclovir hydrochloride as reported in the prior art references involves more than one step thereby making the process lengthy and industrially nonviable. In view of this, there is a need to develop a process for the preparation of valacyclovir hydrochloride, which is simple, cost-effective, efficient and industrially applicable.

The inventors of the present invention have now found that valacyclovir hydrochloride, the compound of formula I can be obtained in good yield and high purity from N-[(benzyloxy)carbonyl]-L-valine-2-[(2-amino-1,6-dihydro-6-oxo-9H-purin-9-yl)methoxy]ethyl ester of formula II through an improved process, which although involves use of a catalyst such as palladium on carbon and a mineral acid for the deprotection of the compound of formula II, the process avoids use of organic solvents, which in-turn reduces the generation of impurities and also renders the process simple and cost-effective. Moreover, the reaction is carried out in a hydogenator using hydrogen pressure ranging from 1 kg/cm² to 15 kg/cm², thereby making the process feasible for large scale manufacturing of valacyclovir hydrochloride. Thus, the present invention provides a simple, cost-effective, efficient and industrially applicable process for the preparation of valacyclovir hydrochloride, which is used for the treatment of genital herpes and herpes zoster.

### OBJECTS OF THE INVENTION

An object of the present invention is to provide a process for the preparation of 2-[(2-amino-1,6-dihydro-6-oxo-9H-purin-9-yl)methoxy]ethyl L-valine ester hydrochloride (valacyclovir hydrochloride) of formula I from N-[(benzyloxy)carbonyl]-L-valine-2-[(2-amino-1,6-dihydro-6-oxo-9H-purin-9-yl)methoxy]ethyl ester (the compound of formula II).

Another object of the present invention is to provide a process for the preparation of valacyclovir hydrochloride in ≧90% yield, and having purity of ≧99.5%, pharmaceutically acceptable grade.

Another object of the present invention is to provide a process for the preparation of valacyclovir hydrochloride using water as a reaction solvent.

Another object of the present invention is to provide a process for the preparation of valacyclovir hydrochloride without involving use of organic solvents.

Yet another object of the present invention is to provide a process for the preparation of valacyclovir hydrochloride of pharmaceutically acceptable grade, without any additional step of purification.

Yet another object of the present invention, the valacyclovir hydrochloride obtained using the process of the present invention is valacyclovir hydrochloride polymorphic Form I.

Still another object of the present invention is to provide a process for the preparation of valacyclovir hydrochloride which is simple and efficient.

Further object of the present invention is to provide a process for the preparation of valacyclovir hydrochloride which is cost-effective and industrially applicable.

### STATEMENT OF THE INVENTION

In accordance with the objects of the present invention there is provided a process for the preparation of 2-[(2-amino-1,6-dihydro-6-oxo-9H-purin-9-yl)methoxy]ethyl L-valine ester hydrochloride (valacyclovir hydrochloride) of formula I from N-[(benzyloxy)carbonyl]-L-valine-2-[(2-amino-1,6-dihydro-6-oxo-9H-purin-9-yl) methoxy]ethyl ester (the compound of formula II) comprising deprotection of a compound of formula II using palladium on carbon as a catalyst and mineral acid in the presence of water as a solvent under the hydrogen pressure to yield valacyclovir hydrochloride.

The process of the present invention is depicted in the following scheme:

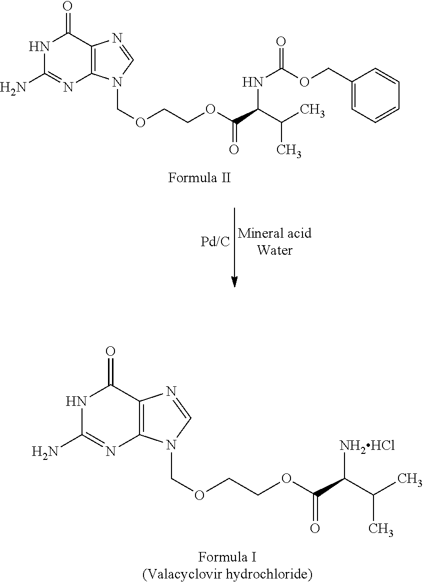

In accordance with another aspect of the present invention, the process for the preparation of valacyclovir hydrochloride provides the compound in a yield of ≧90% and purity of ≧99.5%, pharmaceutically acceptable grade.

In accordance with another aspect of the present invention, the process of the present invention overcomes the disadvantages associated with the process disclosed in the cited prior arts, which concerns with the use of large volume of organic solvents, which in-turn leads to generation of undesired impurities.

In accordance with another aspect of the present invention, the process of the present invention yields valacyclovir hydrochloride of pharmaceutically acceptable grade without any additional step of purification. Valacyclovir hydrochloride obtained by the process of the present invention is characterized as valacyclovir hydrochloride Form I.

In accordance with yet another aspect of the present invention, the process for the preparation of valacyclovir hydrochloride uses water as a reaction solvent, thereby avoiding use of an organic solvent and thus, rendering the process simple, efficient, cost-effective and industrially applicable.

### DESCRIPTION OF THE INVENTION

The present invention relates to a process for preparing 2-[(2-amino-1,6-dihydro-6-oxo-9H-purin-9-yl)methoxy]ethyl L-valine ester hydrochloride (valacyclovir hydrochloride) of formula I,

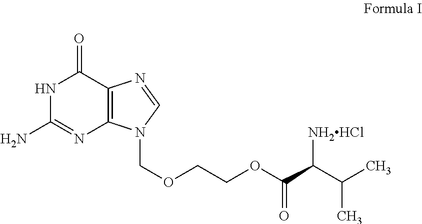

comprising deprotection of N-[(benzyloxy)carbonyl]-L-valine-2-[(2-amino-1,6-dihydro-6-oxo-9H-purin-9-yl)methoxy]ethyl ester, of formula II using 5% palladium on carbon as a catalyst and mineral acid in the presence of water under hydrogen pressure to yield valacyclovir hydrochloride having yield of ≧90% and purity of ≧99.5%, pharmaceutically acceptable grade.

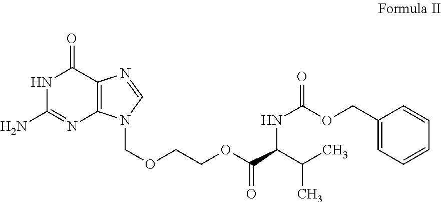 [SMILES: CC(C)[C@H](NC(=O)OCc1ccccc1)C(=O)OCCOCn1cnc2c(=O)[nH]c(N)nc21]

In an embodiment of the present invention, the deprotection of compound of formula II is carried out using 5 to 10 volume of water as a solvent with respect to the compound of formula II.

In accordance with embodiment of the present invention, the deprotection of compound of formula II is carried out using a mineral acid such as hydrochloric acid.

In accordance with embodiment of the present invention, the deprotection of compound of formula II is carried out using hydrochloric acid in 0.10 to 0.25 volume with respect to the compound of formula II.

In accordance with embodiment of the present invention, the deprotection of compound of formula II is carried out using a deprotection catalyst, such as 5% palladium on carbon.

In accordance with embodiment of the present invention, the deprotection of compound of formula II is carried out using a deprotection catalyst in an amount of 5% w/w based on the compound of formula II.

In accordance with embodiment of the present invention, the deprotection of compound of formula II is carried out using a hydrogenator under a hydrogen pressure ranging from 1 kg/cm² to 15 kg/cm², preferably the reaction is carried out under a hydrogen pressure of 3 kg/cm² to 8 kg/cm².

In accordance with embodiment of the present invention, the deprotection of compound of formula II is carried out at a temperature of 25° C. to 30° C.

In accordance with embodiment of the present invention, the deprotection of compound of formula II is carried out in 6 to 12 hours time period.

In accordance with another embodiment of the present invention, valacyclovir hydrochloride of formula I prepared by the process of the present invention involving deprotection of N-[(benzyloxy)carbonyl]-L-valine-2-[(2-amino-1,6-dihydro-6-oxo-9H-purin-9-yl)methoxy]ethyl ester, of formula II using 5% palladium on carbon as a catalyst and mineral acid in the presence of water as a solvent is valacyclovir hydrochloride Form I characterized by X-ray diffraction pattern at about 3.7, 8.6, 10.6, 10.9, 16.5, 24.0 and 27.2±0.2 degrees 20.

In accordance with embodiments of the present invention, the valacyclovir hydrochloride obtained with yield ≧90% and purity of ≧99.5%, pharmaceutically acceptable grade.

In accordance with embodiment of the present invention, the term “pharmaceutically acceptable grade” refers to the valacyclovir hydrochloride of formula I, obtained using the process of the present invention, which contains total organic impurities not more than 5.0%, as disclosed in the U.S. Pharmacopeia 33NF-28.

In accordance with the present invention, the starting material, N-[(benzyloxy)carbonyl]-L-valine-2-[(2-amino-1,6-dihydro-6-oxo-9H-purin-9-yl)methoxy]ethyl ester (the compound of formula II), concentrated hydrochloric acid, 5% palladium on carbon and water are charged to a 1 liter capacity hydrogenator. The reaction mass hydrogenated with hydrogen pressure ranging from 1 kg/cm² to 15 kg/cm² at a temperature of 25-30° C. for 6 to 12 hours. The reaction mass is then filtered under vacuum and washed with water. The filtrate is distilled off under vacuum and then cooled to 30-38° C. To the cooled reaction mass is then added isopropyl alcohol and the reaction mixture is further cooled to a temperature of 5-10° C. and maintained for another 1 hour. The product, valacyclovir hydrochloride obtained is then filtered and washed with isopropyl alcohol to obtain valacyclovir hydrochloride polymorphic Form I having yield of ≧90% and purity of ≧99.5%, pharmaceutically acceptable grade.

The starting material of the process, N-[(benzyloxy)carbonyl]-L-valine-2-[(2-amino-1,6-dihydro-6-oxo-9H-purin-9-yl)methoxy]ethyl ester, (the compound of formula II) is a known compound and can be prepared by a person skilled in the art by following the processes disclosed in the literature. For example, the compound of formula II may be prepared by following the process disclosed in the U.S. Pat. No. 4,957,924, which is incorporated herein by reference. The process involves addition of N-(benzyloxy)carbonyl-L-valine (CBZ-L-valine), dimethylformamide (DMF), 4-dimethylaminopyridine (DMAP) and dicyclohexylcarbodimide (DCC) to the warm solution of acyclovir in DMF to obtain a faint yellow solution. The resulting faint yellow solution was allowed to cool to room temperature and stirred overnight to obtain white precipitate. The reaction mixture was again recharged with the same amounts of CBZ-L-valine, DMAP and DCC, the cloudy suspension obtained is stirred at room temperature for 2 days. The suspension is filtered to remove the solid and the filtrate is concentrated to obtain a light yellow oil. The oil is purified by flash chromatography on silica gel to yield the compound of formula II.

The following examples which fully illustrate the practice of the preferred embodiments of the present invention are intended to be for illustrative purpose only and should not be considered in anyway to limit the scope of the present invention.

### Example 1

To a 1L capacity hydrogenator charged a compound of formula II (100 g), concentrated hydrochloric acid (19.2 ml), 5% palladium on carbon (10 g) and water (700 ml) and hydrogenate the reaction mass under hydrogen pressure of 3 kg/cm² to 8 kg/cm² at a temperature of 25-30° C. for 6 to 12 hours. Filter the reaction mass under vacuum and washed with water. The filtrate was then distilled off under vacuum. The reaction mass was then cooled and isopropyl alcohol was added to the reaction mass. Further, the reaction mass was cooled to a temperature of 5-10° C. and maintained for 1 hour. The product valacyclovir hydrochloride obtained was filtered and washed with isopropyl alcohol.

Yield 90% and purity 99.5%

### Example 2

To a 1L capacity hydrogenator charged a compound of formula II (50 g), concentrated hydrochloric acid (9.6 ml), 5% palladium on carbon (5 g) and water (350 ml) and hydrogenate the reaction mass under hydrogen pressure of 3 kg/cm² to 8 kg/cm² at a temperature of 25-30° C. for 6 to 12 hours. The reaction mass was filtered under vacuum and washed with water. The filtrate was then distilled off under vacuum. The reaction mass was then cooled and isopropyl alcohol was added to the reaction mass. The reaction mass was further cooled to a temperature of 5-10° C. and maintained for 1 hour. The product valacyclovir hydrochloride obtained was filtered and washed with isopropyl alcohol.

Yield 92% and purity 99.8%

### Analytical Method for Analysis

HPLC column: USP L1, 4.6×150 mm, 3.5 μm (Zorbax SB-C18, Part #863953-914)

### Detector: UV 250 nm

Mobile phase A: In a 1000 ml volumetric flask add 2.8 ml of triethylamine, 800 ml of purified water and adjust the pH to 5.00±0.05 with glacial acetic acid. Complete the volume with purified water.

### Mobile Phase B: Acetonitrile

Injection volume: 50 μL
Column temperature: 30° C.
Flow rate: 1.0 mL/minutes

### Gradient

| Time (min) | Mobile Phase A (%) | Mobile Phase B (%) |
|---|---|---|
| 0-12 | 97 | 3 |
| 12-24 | 97 | 3 |
| 24-35 | 70 | 30 |
| 35-36 | 97 | 3 |
| 36-45 | 97 | 3 |

We claim:

1. A process for the preparation of 2-[(2-amino-1,6-dihydro-6-oxo-9H-purin-9-yl)methoxy]ethyl L-valine ester hydrochloride (valacyclovir hydrochloride) of formula I, 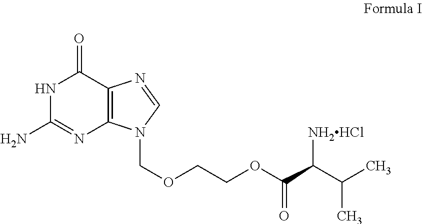
comprising deprotection of N-[(benzyloxy)carbonyl] -L-valine-2- [(2-amino-1,6-dihydro-6-oxo-9H-purin-9-yl)methoxy]ethyl ester, of formula II, 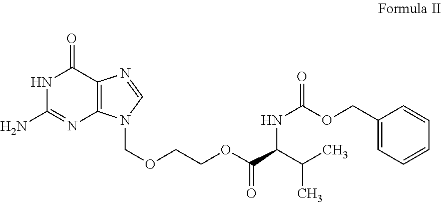 [SMILES: CC(C)[C@H](NC(=O)OCc1ccccc1)C(=O)OCCOCn1cnc2c(=O)[nH]c(N)nc21]
using 5% palladium on carbon as a catalyst and a mineral acid in the presence of water, under hydrogen pressure to obtain valacyclovir hydrochloride, wherein deprotection of the compound of formula II is carried out without use of an organic solvent.

2. The process as claimed in claim 1, wherein said mineral acid used for the deprotection of the compound of formula II is hydrochloric acid.

3. The process as claimed in claim 2, wherein the hydrochloric acid is used in 0.10 to 0.25 volume with respect to the compound of formula II.

4. The process as claimed in claim 1, wherein water is used in 5 to 10 volume with respect to the compound of formula II.

5. The process as claimed in claim 1, wherein the catalyst, 5% palladium on carbon is used in an amount of 5% w/w based on the compound of formula II.

6. The process as claimed in claim 1, wherein said process is carried out at a hydrogen pressure ranging from 1 kg/cm² to 15 kg/cm².

7. The process as claimed in claim 6, wherein said process is carried out at a hydrogen pressure of 3 kg/cm² to 8 kg/cm².

8. The process as claimed in claim 1, wherein the deprotection of compound of formula II is carried out at a temperature of 25° C. to 30° C.

9. The process as claimed in claim 1, wherein said valacyclovir hydrochloride of formula I is valacyclovir hydrochloride Form I.
